SEQUENCE LISTING (22 sequences)

SEQ ID NO: 1 - protein, 9 aa, Artificial Sequence
  Artificially Synthesized Sequence
AFKAWAVAR

SEQ ID NO: 2 - protein, 9 aa, Artificial Sequence
  Artificially Synthesized Sequence
AFKAWAVAR

SEQ ID NO: 3 - protein, 12 aa, Artificial Sequence
  Artificially Synthesized Sequence
AVMDDFAAFVEK

SEQ ID NO: 4 - protein, 6 aa, Artificial Sequence
  Artificially Synthesized Sequence
AWAVAR

SEQ ID NO: 5 - protein, 6 aa, Artificial Sequence
  Artificially Synthesized Sequence
AWAVAR

SEQ ID NO: 6 - protein, 13 aa, Artificial Sequence
  Artificially Synthesized Sequence
DVFLGMFLYEYAR

SEQ ID NO: 7 - protein, 13 aa, Artificial Sequence
  Artificially Synthesized Sequence
DVFLGMFLYEYAR

SEQ ID NO: 8 - protein, 14 aa, Artificial Sequence
  Artificially Synthesized Sequence
DVFLGMFLYEYARR

SEQ ID NO: 9 - protein, 13 aa, Artificial Sequence
  Artificially Synthesized Sequence
DVFLGMFLYEYAR

SEQ ID NO: 10 - protein, 12 aa, Artificial Sequence
  Artificially Synthesized Sequence
RHPDYSVVLLLR

SEQ ID NO: 11 - protein, 8 aa, Artificial Sequence
  Artificially Synthesized Sequence
EFIAWLVK

SEQ ID NO: 12 - protein, 8 aa, Artificial Sequence
  Artificially Synthesized Sequence
EFIAWLVK

SEQ ID NO: 13 - protein, 10 aa, Artificial Sequence
  Artificially Synthesized Sequence
EFIAWLVKGR

SEQ ID NO: 14 - protein, 28 aa, Artificial Sequence
  Artificially Synthesized Sequence
HGEGTFTSDVSSYLEGQAAKEFIAWLVK

SEQ ID NO: 15 - protein, 19 aa, Artificial Sequence
  Artificially Synthesized Sequence
NYAEAKDVFLGMFLYEYAR

SEQ ID NO: 16 - protein, 15 aa, Artificial Sequence
  Artificially Synthesized Sequence
ASGYTFTSYWMHWVR

SEQ ID NO: 17 - protein, 7 aa, Artificial Sequence
  Artificially Synthesized Sequence
DTLMISR

SEQ ID NO: 18 - protein, 24 aa, Artificial Sequence
  Artificially Synthesized Sequence
GLEWVSAITWNSGHIDYADSVEGR

SEQ ID NO: 19 - protein, 24 aa, Artificial Sequence
  Artificially Synthesized Sequence
GLEWVSAITWNSGHIDYADSVEGR

SEQ ID NO: 20 - protein, 14 aa, Artificial Sequence
  Artificially Synthesized Sequence
FNWYVDGVEVHNAK

SEQ ID NO: 21 - protein, 24 aa, Artificial Sequence
  Artificially Synthesized Sequence
GLEWVSAITWNSGHIDYADSVEGR

SEQ ID NO: 22 - protein, 24 aa, Artificial Sequence
  Artificially Synthesized Sequence
GLEWVSAITWNSGHIDYADSVEGR

(19) United States
(12) Patent Application Publication
(10) Pub. No.: US 2021/0187107 A1
(43) Pub. Date: Jun. 24, 2021

(54) METHODS OF PREPARING STABLE LIQUID THERAPEUTIC PROTEIN COMPOSITIONS

(71) Applicant: GLAXOSMITHKLINE INTELLECTUAL PROPERTY DEVELOPMENT LIMITED, Brentford (GB)

(72) Inventors: Wasfi Abdel Karim ALAZZAM, Downingtown, PA (US); Conor Robert LAKE, Malvern, PA (US); Vakhtang Vladimir LOLADZE, Collegeville, PA (US)

(21) Appl. No.: 17/270,746

(22) PCT Filed: Aug. 26, 2019
(86) PCT No.: PCT/IB2019/057158

§ 371 Date: Feb. 23, 2021

Related U.S. Application Data
(60) Provisional application No. 62/724,275, filed on Aug. 29, 2018.

(51) Int. Cl. A61K 39/395

(52) U.S. Cl. CPC A61K 39/39591

(57) ABSTRACT

The invention relates to a method of preparing a stable liquid therapeutic protein pharmaceutical (biopharmaceutical) comprising: a) obtaining an open-ended container comprising a sample, wherein the sample is a therapeutic protein in a solution; b) freezing the sample; c) applying a vacuum while the sample is frozen and; d) sealing the open end of the container while the sample is frozen and while the vacuum is applied to obtain the liquid therapeutic protein composition.

### FIELD OF THE INVENTION

The invention relates to the field of protein pharmaceuticals (biopharmaceuticals). In particular, the invention relates to methods of preparing a stable liquid therapeutic protein composition.

### BACKGROUND OF THE INVENTION

Stability of pharmaceutical protein is a major challenge for developing and manufacturing biopharmaceutics. While there are many types of formulations of protein pharmaceuticals, liquid and lyophilized formulations are among the most common. Liquid formulations are often preferred over lyophilized formulations for reasons related to cost of manufacturing and convenience to health care practitioners and patients. However, liquid formulations often suffer from issues related to stability. Stabilizing excipients in the final liquid formulation are often required to help reduce aggregation, oxidation, color change, to maintain structure, function, and safety of protein pharmaceuticals. Additionally, Iliquid-air interface is one of the main drivers of instabilities for liquid protein formulations. For example, protein pharmaceuticals can be prone to oxidation and to prevent this from occurring, the headspace is often purged with an inert gas, such as N₂ gas, during final drug container filling to promote a longer shelf life.

### SUMMARY OF THE INVENTION

The invention relates to methods of preparing stable liquid therapeutic protein compositions. In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition.

In one embodiment, the therapeutic protein is selected from the group consisting of antigen binding proteins, antibodies, recombinant proteins, fusion proteins, protein domains, enzymes, polypeptides, and protein-drug conjugates.

In another embodiment, the therapeutic protein is a recombinant protein.

In another embodiment, the recombinant protein is a fusion protein.

In yet another embodiment, the therapeutic protein is a monoclonal antibody.

In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition, wherein the solution is a pharmaceutical formulation.

In one embodiment the solution is a pharmaceutical formulation consisting of at least one buffer. In another embodiment the solution is a pharmaceutical formulation, wherein the pharmaceutical formulation does not comprise an excipient.

In one embodiment a method of preparing a liquid therapeutic protein composition in solution comprises:
a. Obtaining a vial comprising a sample, wherein the sample is a therapeutic protein in a solution;
b. Freezing the sample;
c. Applying a vacuum while the sample is frozen; and
d. Sealing the open end of the vial while the sample is frozen and while the vacuum is applied to obtain the liquid therapeutic protein composition.

In one embodiment a method of preparing a liquid therapeutic protein composition comprises:
a. Obtaining an open-ended container comprising a sample, wherein the sample is a therapeutic protein in a solution;
b. Freezing the sample at a temperature below the glass transition temperature of the solution;
c. Applying a vacuum while the sample is frozen; and
d. Sealing the open end of the container while the sample is frozen and while the vacuum is applied to obtain the liquid therapeutic protein composition.

In one embodiment a method of preparing a liquid therapeutic protein composition comprises:
a. Obtaining an open-ended container comprising a sample, wherein the sample is a therapeutic protein in a solution;
b. Freezing the;
c. Applying a vacuum while the sample is frozen is applied until an internal pressure of less than or equal to 150 mTorr is achieved; and
d. Sealing the open end of the container while the sample is frozen and while the vacuum is applied to obtain the liquid therapeutic protein composition.

In one embodiment a method of preparing a liquid therapeutic protein composition comprises:
a. Obtaining an open-ended container comprising a sample, wherein the sample is a therapeutic protein in a solution, wherein the container comprises a headspace between the sample and the open end of the container;
b. Freezing the sample;
c. Applying a vacuum while the sample is frozen; and
d. Sealing the open end of the container while the sample is frozen and while the vacuum is applied to obtain the liquid therapeutic protein composition, wherein after sealing the container the headspace lacks air or is not filled with a gas.

In one embodiment, the obtained liquid therapeutic protein composition has reduced aggregation under stress. In another embodiment, the percent aggregation is reduced to about 5% to about 20% aggregation.

In another embodiment, the obtained liquid therapeutic protein composition has reduced color change under stress. In another embodiment, the liquid therapeutic protein composition has about 2-fold to about 10-fold reduced color change.

In yet another embodiment, the obtained liquid therapeutic protein composition has reduced oxidation under stress. In another embodiment, the percent oxidation is reduced to about 5% to about 20% oxidation.

In one embodiment, the stress is photo stress, thermal stress, and/or mechanical stress.

### BRIEF DESCRIPTION OF THE FIGURES

FIG. 1 demonstrates the effect of photo stress on color change on mAb1, mAb2 and FP1.

FIG. 2 demonstrates the effect of thermal stress on color change on mAb1, mAb2 and FP1.

FIG. 3 demonstrates the effect of photo stress on aggregation of mAb1 using SEC UV.

FIG. 4 demonstrates the effect of photo stress on aggregation of mAb1 using SEC-MALS UV.

FIG. 5 demonstrates the effect of photo stress on aggregation of mAb2 using SEC

UV.

FIG. 6 demonstrates the effect of photo stress on aggregation of mAb2 using SEC-MALS UV.

FIG. 7 demonstrates the effect of photo stress on aggregation of FP1 using SEC UV. FIG. 8 demonstrates the effect of photo stress on aggregation of FP1 using SEC-MALS UV.

FIG. 9 demonstrates the effect of thermal stress on aggregation of FP1 using SEC UV.

### DETAILED DESCRIPTION

It is to be understood that this invention is not limited to particular methods, reagents, compounds, compositions, or biological systems, which can, of course, vary. It is also to be understood that the terminology used herein is for the purpose of describing particular embodiments only, and is not intended to be limiting. As used in this specification and the appended claims, the singular forms “a”, “an”, and “the” include plural referents unless the content clearly dictates otherwise. Thus, for example, reference to “a polypeptide” includes a combination of two or more polypeptides, and the like. “About” as used herein when referring to a measurable value such as an amount, a temporal duration, and the like, is meant to encompass variations of ±20% or ±10%, including ±5%, ±1%, and ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the terminology described herein will be used.

The invention relates to methods of preparing stable liquid therapeutic protein composition. In one embodiment, the stable liquid therapeutic protein compositions are prepared without the need for stabilizing excipients in the liquid formulation buffer. In another embodiment, the stable liquid therapeutic protein compositions are prepared without the need for purging the headspace with an inert gas (i.e. nitrogen) during the filling process. The methods described herein increase stability of the liquid therapeutic protein composition, for example, by reducing aggregation, oxidation, and/or color change to maintain structure and function.

In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition.

### Therapeutic

The term “therapeutic” is used herein to describe the use of a drug, medicine, or active agent (e.g. active agent of a pharmaceutical protein) for the treatment or prevention of a disease or illness. A therapeutic includes (A) articles recognized in the official United States Pharmacopoeia, official Homoeopathic Pharmacopoeia of the United States, or official National

Formulary, or any supplement to any of them; (B) articles intended for use in the diagnosis, cure, mitigation, treatment, or prevention of disease in man or other animals; (C) articles (other than food) intended to affect the structure or any function of the body of man or other animals; and (D) articles intended for use as a component of any article specified in (A), (B), or (C). It will be appreciated by those skilled in the art that references herein to “treatment” refer to the treatment of established conditions. However, “therapeutic” may also include prevention of certain diseases.

### Protein

The term “protein” is used herein in the broadest sense to refer to biomolecules composed of amino acids. “Proteins” include, but are not limited to, antigen binding proteins, antibodies, recombinant proteins, fusion proteins, protein domains, enzymes, polypeptides, and protein-drug conjugates.

The term “antigen binding protein” as used herein refers to antibodies and other protein constructs, such as domains, which are capable of binding to an antigen.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including subclasses of antibodies (e.g. IgG1, IgG2, IgG3, and IgG4), bispecific antibodies, heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

The term “domain” refers to a folded protein structure which retains its tertiary structure independent of the rest of the protein. Generally, domains are responsible for discrete functional properties of proteins and in many cases, may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb(™)” may be considered the same as a “single variable domain”. A single variable domain may be a human single variable domain, but also includes single variable domains from other species such as rodent (for example, as disclosed in WO 00/29004), nurse shark and Camelid VHH dAbs™. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains The term “multi-specific antigen binding protein” refers to antigen binding proteins which comprise at least two different antigen binding sites. Each of these antigen-binding sites will be capable of binding to a different epitope, which may be present on the same antigen or different antigens. The multi-specific antigen binding protein will have specificity for more than one antigen, for example two antigens, or for three antigens, or for four antigens. Examples of “multi-specific antigen binding proteins” include those that consist of, or consist essentially of, an Fc region of an antibody, or a part thereof, linked at each end, directly or indirectly (for example, via a linker sequence) to a binding domain. Such an antigen binding protein may comprise two binding domains separated by an Fc region, or part thereof. By separated is meant that the binding domains are not directly linked to one another, and may be located at opposite ends (C and N terminus) of an Fc region, or any other scaffold region.

The antigen binding protein may comprise two scaffold regions each bound to two binding domains, for example at the N and C termini of each scaffold region, either directly or indirectly via a linker. Each binding domain may bind to a different antigen.

A “dAb™ conjugate” refers to a composition comprising a dAb to which a drug is chemically conjugated by means of a covalent or noncovalent linkage. Preferably, the dAb and the drug are covalently bonded. Such covalent linkage could be through a peptide bond or other means such as via a modified side chain. The noncovalent bonding may be direct (e.g., electrostatic interaction, hydrophobic interaction) or indirect (e.g., through noncovalent binding of complementary binding partners (e.g., biotin and avidin), wherein one partner is covalently bonded to drug and the complementary binding partner is covalently bonded to the dAb™)

When complementary binding partners are employed, one of the binding partners can be covalently bonded to the drug directly or through a suitable linker moiety, and the complementary binding partner can be covalently bonded to the dAb™ directly or through a suitable linker moiety.

As used herein, “dAb™ fusion” refers to a fusion protein that comprises a dAb™ and a polypeptide drug (which could be a dAb™ or mAb). The dAb™ and the polypeptide drug are present as discrete parts (moieties) of a single continuous polypeptide chain.

A “recombinant protein” as used herein refers to a protein indicates that the protein has been modified by the introduction of a heterologous nucleic acid or protein or the alteration of a native nucleic acid or protein. Recombinant proteins may include, for example, fusion proteins. A “fusion protein” is a protein created through the joining of two or more genes that originally coded for separate proteins.

A “protein-drug conjugate” refers to a protein conjugated to one or more drugs such as a cytotoxic agent, a chemotherapeutic agent, a growth inhibitory agent, a toxin (e.g., a protein toxin, an enzymatically active toxin of bacterial, fungal, plant, or animal origin, or fragments thereof), or a radioactive isotope (i.e., a radioconjugate). In one embodiment, the protein drug conjugate is an antibody-drug conjugate (ADC).

In one embodiment a method of preparing a liquid therapeutic protein composition comprises:
a. Obtaining an open-ended container comprising a sample, wherein the sample is a therapeutic protein in a solution, wherein the protein is selected from the group consisting of antigen binding proteins, antibodies, recombinant proteins, fusion proteins, protein domains, enzymes, polypeptides, and protein-drug conjugates;
b. Freezing the sample;
c. Applying a vacuum while the sample is frozen; and
d. Sealing the open end of the container while the sample is frozen and while the vacuum is applied to obtain the liquid therapeutic protein composition.

In another embodiment, the protein is a recombinant protein.

In another embodiment, the recombinant protein is a fusion protein.

In yet another embodiment, the protein is a monoclonal antibody.

### Solution

The “solutions” described herein refer to liquid solutions. In one embodiment, the solution is an aqueous solution. In another embodiment, the solution is a pharmaceutical formulation, i.e. a pharmaceutically acceptable solution capable of being administered to a patient. In another embodiment, the pharmaceutical formulation comprises a buffer and at least one excipient. A “buffer solution” as used herein refers to a solution that resists changes in pH when acid or alkali is added to it. Buffer solutions typically involve a week acid or alkali together with one of its salts. Exemplary buffer solutions include, but are not limited to, salts, sodium acetate, sodium phosphate, sodium citrate, histidine, and phosphate buffered saline (PBS).

An “excipient” as used herein refers to a substance formulated along with the buffer and the active ingredient or drug (e.g. therapeutic protein) and is included for various purposes such as, for example, stabilizing the active ingredient, bulking up the solution, or providing a therapeutic enhancement. Classes of excipients include, for example, chelating agents, surfactants, amino acids, sugars, tonicity modifiers, and cryoprotectants. Pharmaceutical Technology, Aug. 15, 2015, Vol. 2015 Supplement, Issue 3, pg. s35-s39.

Chelating agents sequester metal ions in solution in order to protect the therapeutic proteinfrom degradation (e.g., oxidation). Exemplary chelating agents include EDTA and histidine.

Surfactants are common stabilizers, often added to protein pharmaceutical formulations. Exemplary surfactants include non-ionic detergents such as polysorbates (e.g. polysorbate 20, polysorbate 80, Tween 20)

Amino acids are also common stabilizers for protein pharmaceutical formulations. Exemplary amino acids include arginine, glycine, histidine, and methionine.

Sugars are often added in protein pharmaceutical formulation to reduce aggregation of the protein active ingredient. Exemplary sugars include mannitol, sorbitol, trehalose, methyl R-D-mannopyranoside, lactose, sucrose, and cellobiose

Additional salts can be added to the pharmaceutical formulation to enhance stability. Additional salts include sodium chloride and sodium citrate.

Pharmaceutical formulations may, for example, consist only of a buffer solution, i.e., they do not contain any excipients.

In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition, wherein the solution is a pharmaceutical formulation.

In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition,
   wherein the solution is a pharmaceutical formulation consisting of a buffer solution.

In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition,
   wherein the solution is a pharmaceutical formulation, and wherein the pharmaceutical formulation does not comprise an excipient.

### Container

A “container” refers to any vessel capable of retaining a liquid having at least one open-ended side for liquid filling, and is capable of withstanding low temperatures (e.g. temperatures at or below 0 degrees Celsius). A “final container” is the immediate unit, bottle, vial, ampule, tube, or other receptacle containing the product as distributed for sale, barter, or exchange.

Ideally, a stopper or cap can be applied to the open-ended portion of the container to seal the container and maintain a vacuum. Exemplary containers include vials, bottles, tubes, and syringes. A container can be made from various materials, including, for example, glass or plastic. In one embodiment the container is a vial with a stopper that allows for air exchange during application of the vacuum before the container is sealed.

In one embodiment a method of preparing liquid therapeutic protein composition in solution comprises:
a. Obtaining a vial comprising a sample, wherein the sample is a therapeutic protein in a solution;
b. Freezing the sample;
c. Applying a vacuum while the sample is frozen; and
d. Sealing the open end of the vial while the sample is frozen and while the vacuum is applied to obtain the liquid therapeutic protein composition.

### Freezing

The “freezing” temperature described herein refers to a temperature at or below the glass transition temperature of the entire solution, including formulation buffers, excipients and the protein. “Freezing” temperatures can include temperatures at or below 0 degrees Celsius. In one embodiment, the freezing temperature is below about −50 degrees Celsius. In another embodiment, the freezing temperature is about 0 degrees Celsius, about −10 degrees Celsius, about −20 degrees Celsius, about −30 degrees Celsius, about −40 degrees Celsius, about −50 degrees Celsius, about −60 degrees Celsius, about −70 degrees Celsius, or about −80 degrees Celsius.

The liquid therapeutic protein composition may be maintained frozen, thawed to room temperature, or stored at above freezing but below room temperature, until administration to a patient in need thereof.

In one embodiment a method of preparing a liquid therapeutic protein composition comprises:
a. Obtaining an open-ended container comprising a sample, wherein the sample is a therapeutic protein in a solution;
b. Freezing the sample at a temperature below the glass transition temperature of the solution;
c. Applying a vacuum while the sample is frozen; and
d. Sealing the open end of the container while the sample is frozen and while the vacuum is applied to obtain the liquid therapeutic protein composition.

In one embodiment a method of preparing a liquid therapeutic protein composition comprises:
a. Obtaining an open-ended container comprising a sample, wherein the sample is a therapeutic protein in a solution;
b. Freezing the sample at a temperature below the glass transition temperature of the solution;
c. Applying a vacuum while the sample is frozen;
d. Sealing the open end of the container while the sample is frozen and while the vacuum is applied to obtain liquid therapeutic protein composition; and
e. Thawing the sample prior to administration to a patient.

### Vacuum

“Vacuum” refers to the space in which the pressure is lower than atmospheric pressure. In one embodiment, an internal pressure is applied until near total vacuum is reached. In another embodiment, a vacuum is applied until the internal pressure reaches 150 mTorr or less. In another embodiment, a vacuum is applied until the internal pressure reaches 100 mTorr or less.

In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen until an internal pressure of less than or equal to 150 mTorr is achieved; and
d. Sealing the open end of the container while the sample is frozen and while the vacuum is applied to obtain the liquid therapeutic protein composition.

### Headspace

“Headspace” refers to the space in the container that is not occupied by the sample. For example, in a vial or a bottle, the headspace is the volume between the liquid sample and the top of the container. In one embodiment, after preparing the liquid therapeutic protein composition, the headspace lacks any type of atmosphere. In another embodiment, preparation of the liquid therapeutic protein composition does not comprise filling the headspace with a gas (e.g., nitrogen), i.e., the air or vacuum in the headspace is not replaced with a gas.

In one embodiment a method of preparing a liquid therapeutic protein composition comprises:
a. Obtaining an open-ended container comprising a sample, wherein the sample is a therapeutic protein in a solution, wherein the container comprises a headspace between the sample and the open end of the container;
b. Freezing the sample;
c. Applying a vacuum while the sample is frozen; and
d. Sealing the open end of the container while the sample is frozen and while the vacuum is applied to liquid therapeutic protein composition, wherein after sealing the container the headspace lacks air or is not filled with a gas.
   Sealing “Sealing” refers to closing the open-ended portion of the container with a container closure system so as to contain the liquid in the container, maintain the vacuum, and protect the liquid therapeutic protein composition against contamination and other environmental factors. Container closure systems include, for example, seals, screw caps, stoppers (e.g. rubber), and/or crimp caps (e.g. aluminium).

In one embodiment, sealing is accomplished by closing the open-ended portion of a container with a stopper. In another embodiment, the stopper has a channel for air exchange. In another embodiment, sealing is accomplished by closing the open-ended portion of a container with a stopper, followed by capping the stopper. The stoppers commonly used for protein drug products are typically vented, 2-legged or 3-legged designs. In one embodiment a method of preparing a liquid therapeutic protein composition comprises:
a. Obtaining an open-ended container comprising a sample, wherein the sample is a therapeutic protein in a solution;
b. Freezing the sample;
c. Applying a vacuum while the sample is frozen; and
d. Sealing the open end of the container with a stopper while the sample is frozen and while the vacuum is applied to obtain the liquid therapeutic protein composition.

### Stability

Chemical stability of a liquid therapeutic protein composition is a matter of great concern as it affects the safety and efficacy of the drug product. Stability of a liquid therapeutic protein composition is often a challenge for developing and manufacturing biopharmaceutics. Various protein instability issues, such as aggregation, oxidation, color change, higher order structure, and binding need to be addressed during liquid therapeutic protein composition development.

Protein “aggregation” refers to many types of molecular assemblies. Aggregation can arise from noncovalent interactions or from covalently linked species. Protein aggregation is a frequently cited challenge in the manufacturing and development of protein therapeutics. The major concerns with aggregation are loss of efficacy, receptor activation through cross-linking and immunogenicity (Pharmaceutical Research, Vol. 27, No. 4, Apr. 2010 (MABS 2018, Vol. 10, No.4, 513-538). Solutions for reducing aggregation include, but are not limited to, the use of excipients, manufacturing processes and the container or closure used for the final protein pharmaceutical product. (The AAPS Journal 2006; 8 (3) Article 66).

Protein aggregations typically has a different molecular mass than a protein monomer. Therefore, one way to measure aggregates species is by size exclusion chromatography (SEC). In this method, the protein sample is applied to a column made of resins with certain pore sizes. The smaller molecular mass species, such as fragments and monomer, elute earlier than aggregates. The different eluting species are measured with a UV diode array detector absorbance at 280 nm to specifically quantify the amounts of protein's different molecular mass species present in the sample. Another method for measuring protein aggregation is by using a multi-angle light scattering (MALS) detector to detect and measure molecular mass of aggregates.

“Color change” or “coloration change” refers to a visual change in color of the liquid pharmaceutical product. Coloration changes may indicate chemical modifications of the products. Product coloration variability can not only affect the assessment of the comparability of the pre- and post-change materials, but it can also affect clinical development, particularly the ability to blind clinical trials involving placebo controls. Color control has been a major challenge for liquid formulation of protein therapeutics. Changing processing parameters and excipients are typical solutions. (MABS 2018, Vol. 10, No.4, 513-538).

Color can be measured by visual appearance inspection, to compare the color of the product to an existing color standard by eye. Color can also be measured using calibrated instrumentation, which quantitatively measures the distance of the sample color from the color of a standard. In this method, the results are expressed as ΔE (distance of color from standard). “Oxidation” refers to a chemical reaction in which electrons are lost. Oxidation is one of the major chemical degradation pathways for protein pharmaceuticals. Methionine, cysteine, histidine, tryptophan, and tyrosine are the amino acid residues most susceptible to oxidation due to their high reactivity with various reactive oxygen species. Oxidation during protein processing and storage can be induced by contaminating oxidants, catalyzed by the presence of transition metal ions and induced by light. Protein oxidation may result in loss of biological activity and other undesirable pharmaceutical consequences. (Biotechnology and Engineering, Vol. 48, No. 5, Dec. 5, 1995). Oxidation is often measured by peptide map mass spectrometry.

### Stress

“Stress” refers to any internal or external pressures that effect or alter the stability of the liquid therapeutic protein composition. A “stress” includes, for example, photo stress (light), thermal stress (temperature), mechanical stress (physical agitation), or combinations thereof. Forced degradation studies, also known as stress testing, are processes that involve degradation of drug products and drug substances at conditions more severe than typical conditions and thus generates degradation products that can be studied to determine the stability of the molecule. (Journal of Pharmaceutical Analysis, Vol. 4, Issue 3, Pages 159-165, Sep. 17, 2013).

Photostability studies of drugs and drug products are an integral part of the product development process in the pharmaceutical industry. As used herein, “photo stress” or “light stress” refers to the exposure of the liquid therapeutic protein composition to solar, UV, and/or visible light. The resulting physical and/or chemical changes (i.e. stability) of the pharmaceutical protein can be measured (e.g. aggregation, oxidation and/or color change). (International Journal of Photoenergy, Vol. 2016, Article ID 8135608, 2016). The ICH guideline Q1B(1)(C) specifies the experimental setup for photostability studies, including light sources and extent of light exposure. The product should be exposed to at least 1.2 million lux hours in the visible range (400-800 nm) and at least200Wh/m2. (Journal of Pharmaceutical Sciences, Vol. 101, NO. 3, Mar. 2012).

As used herein, “thermal stress” refers to exposing the liquid therapeutic protein composition to temperatures higher than the recommended storage temperature. With increasing temperature, proteins may undergo conformational changes such as unfolding or partial unfolding. These changes may subsequently lead to other degradation reactions, including aggregation. A suitable temperature for thermal stress testing needs to be selected on a case-by-case approach. For example, for a drug product intended for storage at 2-8° C., accelerated testing is often performed at 25° C., as laid down in ICH Q1A(R2). Thermal stress conditions can also include temperatures at 40° C. at 75% relative humidity for 4 weeks. (Journal of Pharmaceutical Sciences, Vol. 101, NO. 3, Mar. 2012).

As used herein, “mechanical stress” refers to physical manipulation including, for example, agitation (shaking), stirring, pumping, vortexing, sonication, or shearing for a specified time.

The various forms of stress (forced degradation studies) can be applied alone, or in combination. When applied in combination, multiple forms of stress can occur simultaneous or sequentially.

In one embodiment, the methods described herein comprise obtaining a liquid therapeutic protein composition that has reduced aggregation under stress. In another embodiment, the methods described herein comprise obtaining a liquid therapeutic protein composition wherein the percent aggregation of the protein is reduced to about 0.01% to about 20%, about 0.1% to about 10%, about 1% to about 20%, about 1% to about 10%, for example about 1%, about 2%, about 3%, about 4%, about 5%, about 10%, about 15%, or about 20% total percent aggregation.

In one embodiment, the methods described herein comprise obtaining a liquid therapeutic protein composition that has reduced aggregation under photo stress.

In one embodiment, the methods described herein comprise obtaining liquid therapeutic protein composition that has reduced aggregation under thermal stress.

In one embodiment, the methods described herein comprise obtaining a liquid therapeutic protein composition that has reduced aggregation under mechanical stress.

In one embodiment, the methods described herein comprise obtaining a liquid therapeutic protein composition that has reduced color change under stress. In another embodiment, the methods described herein comprise obtaining a liquid therapeutic protein composition that has about 2-fold, about 3-fold, about 4-fold, about 5-fold, about 6-fold, about 7-fold, about 8-fold, about 9-fold, or about 10-fold reduced color change under stress.

In one embodiment, the methods described herein comprise obtaining a liquid therapeutic protein composition that has reduced color change under photo stress.

In one embodiment, the methods described herein comprise obtaining a liquid therapeutic protein composition that has reduced color change under thermal stress.

In one embodiment, the methods described herein comprise obtaining a liquid therapeutic protein composition that has reduced color change under mechanical stress.

In one embodiment, the methods described herein comprise obtaining a liquid therapeutic protein composition that has reduced oxidation or dioxidation under stress. In another embodiment, the methods described herein comprise obtaining a liquid therapeutic protein composition wherein the percent oxidation or dioxidation of the protein is reduced to about 0.01% to about 20%, about 0.1% to about 10%, about 1% to about 20%, about 1% to about 10%, for example about 1%, about 2%, about 3%, about 4%, about 5%, about 10%, about 15%, or about 20%.

In one embodiment, the methods described herein comprise obtaining a liquid therapeutic protein composition that has reduced oxidation under photo stress.

In one embodiment, the methods described herein comprise obtaining a liquid therapeutic protein composition that has reduced oxidation under thermal stress.

In one embodiment, the methods described herein comprise obtaining a liquid therapeutic protein composition that has reduced oxidation under mechanical stress.

### EXAMPLES

### Materials and Solutions

All chemicals were purchased from Sigma-Aldrich Chemical Co. (St. Louis, Mo.).

Solutions were made by dissolving chemicals in deionized water that was further purified using a Millipore Milli-Q system.

### Liquid Protein Samples

Table 1 describes the various formulation conditions. “Native formulation” refers to the full formulation which includes a buffer solution and excipients. “Modified formulation” refers to a simple formulation in which all excipients were removed and only contains a buffer solution.

TABLE 1

| Protein | Native Formulation | Modified Formulation |
|---|---|---|
| Monoclonal | Sodium acetate + 4 excipients | Sodium acetate only |
| antibody 1 | (chelating agent, surfactant, | |
| (mAb1) | amino acid, and additional salt) | |
| Monoclonal | Sodium acetate + 3 excipients | Sodium acetate only |
| antibody 2 | (chelating agent, surfactant, and | |
| (mAb2) | additional salt) | |
| Fusion | Sodium phosphate + 3 excipients | Sodium phosphate |
| Protein 1 | (2 sugars and a surfactant) | only |
| (FP1) | | |

### Preparation of Vacuum Headspace and Control Samples

Samples were prepared in a lyophilizer (LyoStar3 by SP Scientific with Praxair Control Lyo Technology S/N:319654) to utilize the temperature and vacuum controls. Samples were not lyophilized at any time. Liquid protein samples were placed in a vial and were frozen to −70° C. Once the temperature was reached and stabilized at −70° C., a vacuum was pulled until total vacuum (<150 mTorr) was reached. Once the vacuum reached <150 mTorr, the vials were capped with stopper and sealed with an aluminium cap. These samples may be referred to as “Vacuumed headspace” or “Vacuumed HS”.

Control samples were also obtained. The control samples were frozen but did not undergo the vacuum headspace preparation and were subjected to the following stress conditions. Controls samples were used to identify baseline degradation (aggregation, color change, and/or oxidation). These samples may be referred to as “Control”, “Atm” or “normal Atm”.

### Photo Stress Conditions

Samples, including the control samples and those that underwent the vacuum headspace procedure, were thawed and transferred to a photo stability chamber (CARON PhotoStability Chamber model 6545-2) and stressed to 1 ICH unit of photo stress which equates to 1.2 million lux hours of visible light and 200 w/m² of UV light. The stability chamber was set to 25° C. and 60% relative humidity.

### Thermal Stress Conditions

Samples, including the control samples and those that underwent the vacuum headspace procedure, were thawed and transferred to a thermal stability chamber (CARON Thermostability chamber) and stressed at 40° C/75% relative humidity for 4 Weeks.

### Example 1

### Stability of Color Change (Effect of Photo Stress and Thermal Stress on Color Change)

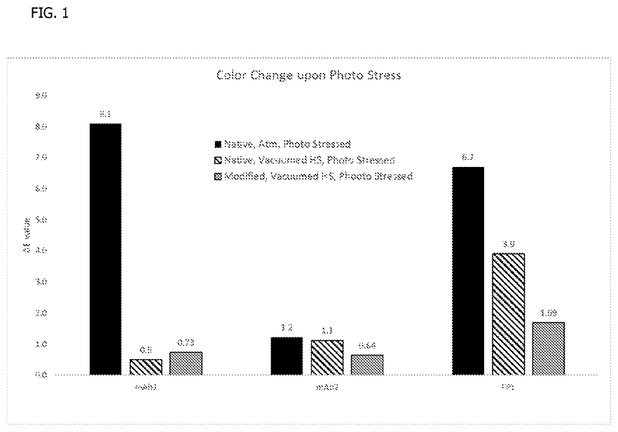

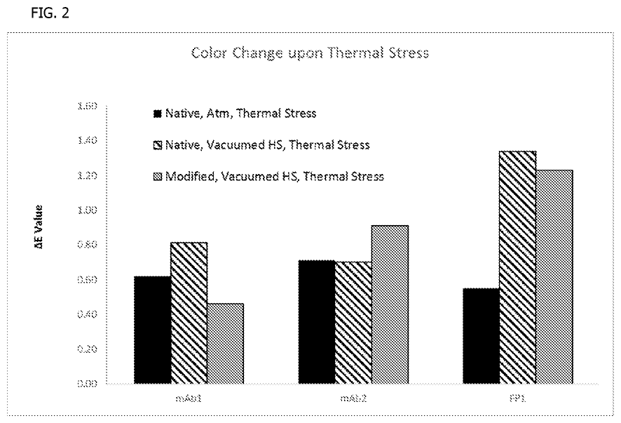

After photo stress or thermal stress was completed, the samples were measured for color change using the Hunter Ultrascan VIS. The color of each sample was measured and compared to a no stress control to quantify the amount of color change following the photo stress via the ΔE distance formula. The other measured parameters are part of the L,a,b color space. These values form a coordinate in a three-dimensional color space. The ΔE distance formula calculates the space between two coordinates in the L,a,b color space. A ΔE distance greater than 2.0 is said to be significantly different from a referenced color. Results are reproduced in graphical form in FIG. 1, FIG. 2, and numerical form in Table 2, and demonstrate that samples that have undergone the vacuumed headspace methods described herein, have reduced color change upon photo stress and/or thermal stress. The ΔE calculation is a measurement of distance between two separate L,a,b values in the 3-dimensional CIELAB color space. This calculation is used as a comparison tool to determine the nearest neighbour for when measuring an unknown to a reference value; the smallest value would be matched. The calculation yields the total color difference from the control or “no stress” sample. Any ΔE calculation higher than 2.0 was considered a significant color change that would be easily perceptible with human vision.

TABLE 2

| | | Headspace | EP Color | EP Color | EP Color |
|---|---|---|---|---|---|
| Stress Condition | Buffer | Condition | Match mAb1 | Match mAb2 | Match FP1 |
| Control | Native | Normal Atm | EP B4.9 | EP B4.0 | EP Y0.1 |
| Control | Native | Vacuumed HS | EP B4.9 | EP B4.0 | EP Y0.1 |
| Control | Modified | Vacuumed HS | EP B5.4 | EP B4.7 | EP Y1.0 |
| Photo (1 xICH) | Native | Normal Atm | EP BY2.5 | EP B3.7 | EP B0.1 |
| Photo (1 xICH) | Native | Vacuumed HS | EP B4.9 | EP BY3.8 | EP Y1.2 |
| Photo (1 xICH) | Modified | Vacuumed HS | EP BY5.2 | EP B4.8 | EP Y0.4 |
| Thermal (40° C. | Native | Normal Atm | EP BY4.7 | EP B4.0 | EP Y0.1 |
| for 4 Weeks) | | | | | |
| Thermal (40° C. | Native | Vacuumed HS | EP B4.7 | EP B3.9 | EP BY0.1 |
| for 4 Weeks) | | | | | |
| Thermal (40° C. | Modified | Vacuumed HS | EP BY5.3 | EP B4.6 | EP BY1.2 |
| for 4 Weeks) | | | | | |

### Example 2

### Stability of Aggregation (Effect of Photo Stress and Thermal Stress on Aggregation)

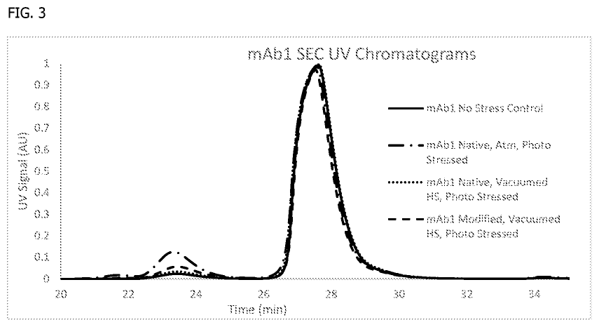

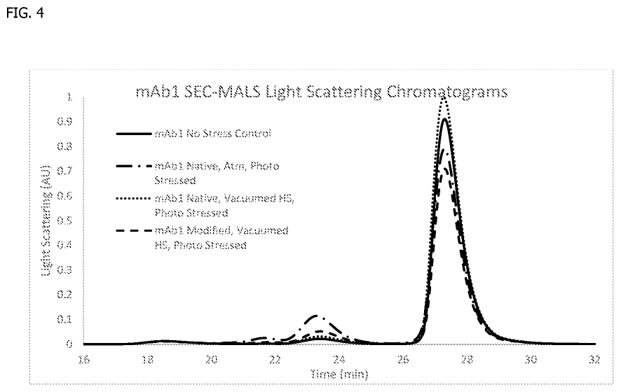

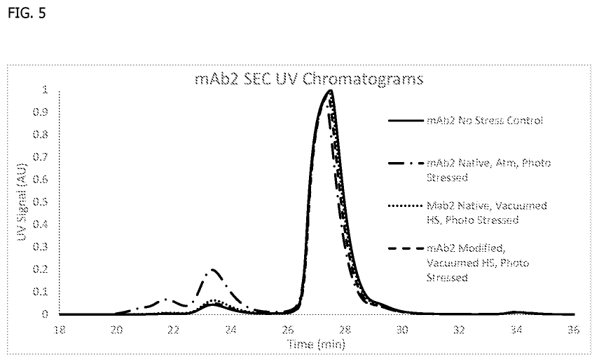

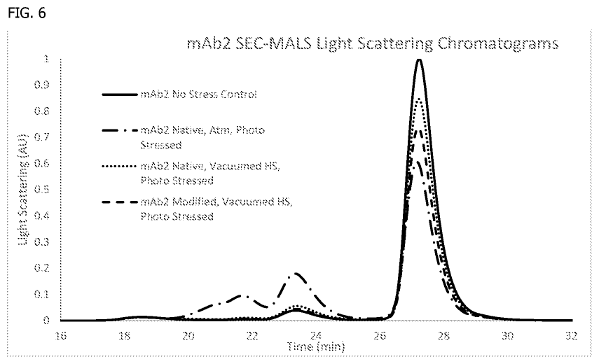

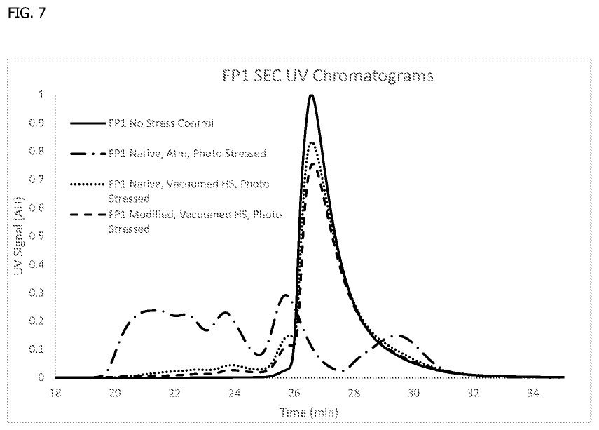

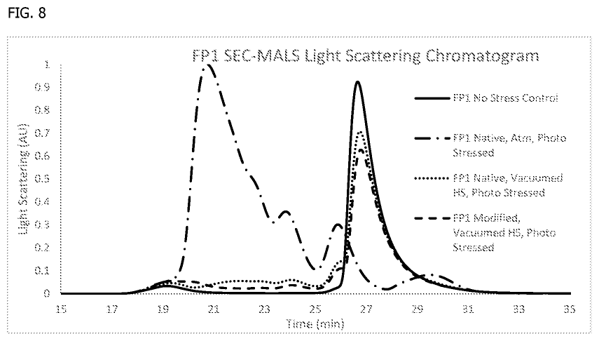

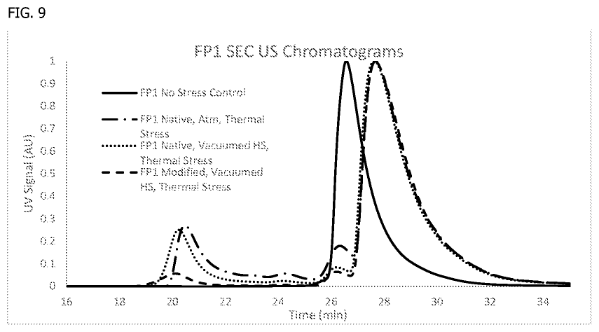

After photo stress or thermal stress was completed, the samples were measured for aggregation via SEC UV chromatography and SEC-MALS light scattering chromatography (Wyatt DAWN HELOES II on an Agilent 1100 series HPLC system). The results of the photo stressed samples are reproduced in Table 3 and FIGS. 3-8. The results of the thermal stressed samples are reproduced in Table 4 and FIG. 9. This data demonstrates that samples that have undergone the vacuumed headspace methods described herein, have reduced aggregation upon photo stress and/or thermal stress.

TABLE 3
Effect of photo stress on aggregation

| Protein | Condition | % Aggregate |
|---|---|---|
| mAb1 | No Stress Control | 1.6 |
| | Native, Atm, Photo Stress | 8.8 |
| | Native, Vacuumed HS, Photo Stress | 1.3 |
| | Modified, Vacuumed HS, Photo Stress | 4.8 |
| mAb2 | No Stress Control | 2.4 |
| | Native, Atm, Photo Stress | 19.1 |
| | Native, Vacuumed HS, Photo Stress | 4.3 |
| | Modified, Vacuumed HS, Photo Stress | 3.8 |
| FP1 | No Stress Control | 0.4 |
| | Native, Atm, Photo Stress | 98.1 |
| | Native, Vacuumed HS, Photo Stress | 15.0 |
| | Modified, Vacuumed HS, Photo Stress | 11.4 |

TABLE 4
Effect of thermal stress on aggregation

| Protein | Condition | % Aggregate |
|---|---|---|
| mAb1 | No Stress Control | 1.6 |
| | Native, Atm, Thermal Stress | 1.8 |
| | Native, Vacuumed HS, Thermal Stress | 1.5 |
| | Modified, Vacuumed HS, Thermal Stress | 2.3 |
| mAb2 | No Stress Control | 2.4 |
| | Native, Atm, Thermal Stress | 3.2 |
| | Native, Vacuumed HS, Thermal Stress | 3.0 |
| | Modified, Vacuumed HS, Thermal Stress | 2.5 |
| FP1 | No Stress Control | 0.4 |
| | Native, Atm, Thermal Stress | 26.2 |
| | Native, Vacuumed HS, Thermal Stress | 18.2 |
| | Modified, Vacuumed HS, Thermal Stress | 7.4 |

### Example 3

### Stability of Oxidation (Effect of Photo Stress and Thermal Stress on Oxidation)

After photo stress or thermal stress was completed, the samples were measured for oxidation or dioxidation via mass spectrometry peptide mapping. The results are reproduced in Tables 5-10. Tables 5, 6, 7, and 9 demonstrate percent oxidation of tryptophan and methionine oxidation modifications. Tables 8 and 10 represent total percent oxidation of the entire protein. These results demonstrate that samples that have undergone the vacuumed headspace methods described herein, have reduced oxidation and/or dioxidation upon photo stress and/or thermal stress.

TABLE 5
Effect of vacuumed headspace on oxidation of FP1 under photo stress

| | | % Modification | | | |
|---|---|---|---|---|---|
| | | | Photo | Photo | |
| | | | Stress | Stress | Photo Stress |
| | | | (1 × ICH) | (1 × ICH) | (1 × ICH) |
| | | | Native | Native | Modified |
| FP1 | | | Buffer | Buffer | Buffer |
| Sequence | Modification | Control | Normal Atm | Vacuum | Vacuum |
| AFKAWAVAR | Dioxidation | 0.0 | 34.6 | 1.5 | 3.2 |
| AFKAWAVAR | Oxidation | ND | 64.6 | ND | ND |
| AVMDDFAAFVEK | Oxidation | 2.4 | 28.0 | 1.8 | 2.6 |
| AWAVAR | Dioxidation | 0.1 | 38.0 | 0.5 | 0.6 |
| AWAVAR | Oxidation | 0.1 | 4.9 | 0.6 | 0.5 |
| DVFLGMFLYEYAR | Dioxidation | 0.0 | 2.0 | 0.1 | 0.0 |
| DVFLGMFLYEYAR | Oxidation | 19.6 | 78.1 | 26.9 | 22.3 |
| DVFLGMFLYEYARR | Oxidation | 2.6 | 23.0 | 2.6 | 2.3 |
| DVFLGMFLYEYAR | Oxidation | 4.7 | 46.7 | 5.8 | 5.3 |
| RHPDYSWLLLR | | | | | |
| EFIAWLVK | Dioxidation | 0.1 | 22.3 | 0.4 | 0.4 |
| EFIAWLVK | Oxidation | 24.4 | 88.1 | 26.6 | 29.1 |
| EFIAWLVKGR | Oxidation | ND | 6.4 | ND | ND |
| HGEGTFTSDVSSYLEG | Dioxidation | ND | 18.9 | 0.2 | 0.5 |
| QAAKEFIAWLVK | | | | | |
| NYAEAKDVFLGMFLYEYAR | Oxidation | 0.0 | 7.4 | 0.8 | 0.7 |

TABLE 6
Effect of vacuumed headspace on oxidation
of mAb2 under photo stress

| | | % Modification | | |
|---|---|---|---|---|
| | | | 1 × ICH | 1 × ICH |
| | | | Native | Mod |
| mAb2 | | | Buffer | Buffer |
| Sequence | Modification | Control | Vacuum | Vacuum |
| ASGYTFTS | Oxidation | 0.1 | 1.1 | 0.2 |
| YWMHWVR | | | | |
| DTLMISR | Oxidation | 2.7 | 4.7 | 6.8 |

TABLE 7
Effect of vacuumed headspace on oxidation
of mAbl under photo stress

| | | % Modification | | |
|---|---|---|---|---|
| | | | 1 × | |
| | | | ICH | 1 × |
| | | | Native | ICH |
| | | | Buffer | Native |
| mAb1 | | | Normal | Buffer |
| Sequence | Modification | Control | Atm | Vacuum |
| GLEWVSAI | Dioxidation | 0.1 | 4.4 | 0.4 |
| TWNSGHID | | | | |
| YADSVEGR | | | | |
| GLEWVSAI | Oxidation | 0.0 | 3.1 | 0.5 |
| TWNSGHID | | | | |
| YADSVEGR | | | | |

TABLE 8
Effect of photo stress on overall oxidation scores

| | | Oxidation | Relative |
|---|---|---|---|
| Protein | Condition | Score | Oxidation |
| mAb1 | Control | 0.1 | 1.0 |
| | Normal Headspace 1X ICH | 7.5 | 98.0 |
| | Vacuum Headspace 1X ICH | 0.9 | 12.1 |
| mAb2 | Control | 58.4 | 1.0 |
| | Vacuum Headspace 1X ICH | 58.3 | 1.0 |
| FP1 | Control | 54.0 | 1.0 |
| | Normal Headspace 1X ICH | 463.0 | 8.6 |
| | Vacuum Headspace 1X ICH | 67.8 | 1.3 |

TABLE 9
Effect of vacuumed headspace on oxidation of mAb1 under thermal stress

| | | % Modification | | | |
|---|---|---|---|---|---|
| | | | 4 wks 40° C. | 4 wks 40° C. | 4 wks 40° C. |
| mAb1 | | | Native | Native | Modified |
| Sequence | Modification | Control | Normal Atm | Vacuum | Vacuum |
| FNWYVDGVEVHNAK | Oxidation | 0.0 | 0.3 | 3.1 | 2.3 |
| GLEWVSAITWNSGHIDYADSVEGR | Dioxidation | 0.2 | 0.9 | 0.8 | 0.7 |
| GLEWVSAITWNSGHIDYADSVEGR | Oxidation | 0.4 | 0.8 | 1.5 | 1.1 |

TABLE 10
Effect of thermal stress on overall oxidation scores

| | | Oxidation | Relative |
|---|---|---|---|
| Protein | Condition | Score | Oxidation |
| mAb1 | Control | 1.0 | 1.0 |
| | 4 weeks 40° C., Native Buffer, | 2.2 | 2.3 |
| | Normal Atm | | |
| | 4 weeks 40° C., Native Buffer, | 5.9 | 6.1 |
| | Vacuum | | |
| | 4 weeks 40° C., Modified Buffer, | 4.5 | 4.6 |
| | Vacuum | | |
| mAb2 | Control | 47.7 | 1.0 |
| | 4 weeks 40° C., Native Buffer, | 52.0 | 1.1 |
| | Normal Atm | | |
| | 4 weeks 40° C., Native Buffer, | 46.3 | 1.0 |
| | Vacuum | | |
| | 4 weeks 40° C., Modified Buffer, | 51.2 | 1.1 |
| | Vacuum | | |
| FP1 | Control | 50.8 | 1.0 |
| | 4 weeks 40° C., Native Buffer, | 68.9 | 1.4 |
| | Normal Atm | | |
| | 4 weeks 40° C., Native Buffer, | 56.4 | 1.1 |
| | Vacuum | | |
| | 4 weeks 40° C., Modified Buffer, | 55.2 | 1.1 |
| | Vacuum | | |

1. A method of preparing a liquid therapeutic protein composition comprising:
a. Obtaining an open-ended container comprising a sample, wherein the sample is a therapeutic protein in a solution;
b. Freezing the sample;
c. Applying a vacuum while the sample is frozen; and
d. Sealing the open end of the container while the sample is frozen and while the vacuum is applied to obtain the liquid therapeutic protein composition.

2. The method of claim 1, wherein the therapeutic protein is selected from the group consisting of antigen binding proteins, antibodies, recombinant proteins, fusion proteins, protein domains, enzymes, polypeptides, and protein-drug conjugates.

3. The method of claim 1, wherein the therapeutic protein is a recombinant protein.

4. The method of claim 3, wherein the recombinant protein is a fusion protein.

5. The method of claim 1, wherein the therapeutic protein is a monoclonal antibody.

6. The method of claim 1, wherein the solution is a pharmaceutical formulation.

7. The method of claim 6, wherein the pharmaceutical formulation consists of at least one buffer.

8. The method of claim 6, wherein the pharmaceutical formulation does not comprise an excipient.

9. The method of claim 1, wherein the container is a vial.

10. The method of claim 1, wherein the freezing temperature is below the glass transition temperature of the solution.

11. The method of claim 1, wherein the vacuum is applied until an internal pressure of less than or equal to 150 mM Ton is achieved.

12. The method of claim 1, wherein the container comprises a headspace between the sample and the open end of the container; and wherein after sealing the container the headspace lacks air or is not filled with a gas.

13. The method of claim 1, wherein the container is sealed with a stopper.

14. The method of claim 1, wherein the obtained liquid therapeutic protein composition has reduced aggregation under stress.

15. The method of claim 14, wherein the aggregation is reduced to about 5% to about 20%.

16. The method of claim 1 wherein the obtained liquid therapeutic protein composition has reduced color change under stress.

17. The method of claim 16, wherein the color change is reduced about 2-fold to about 10-fold.

18. The method of claim 1, wherein the obtained liquid therapeutic protein composition has reduced oxidation under stress.

19. The method of claim 18, wherein the oxidation is reduced to about 5% to about 20%.

20. The method of claim 14, wherein the stress is at least one selected from the group consisting of photo stress, thermal stress, and mechanical stress.
